(19) United States
(12) Patent Application Publication
(10) Pub. No.: US 2021/0214306 A1
(43) Pub. Date: Jul. 15, 2021

(54) PROCESS FOR PREPARATION OF FOSNETUPITANT

(71) Applicant: CADILA HEALTHCARE LIMITED, Sarkhej-Gandhinagar Hwy (IN)

(72) Inventors: Kumar Kamlesh SINGH, Ahmedabad (IN); Sanjay Jagdish DESAI, Ahmedabad (IN); Jagdish Maganbhai PATEL, Ahmedabad (IN); Chirag Jayantilal VYAS, Ahmedabad (IN); Dhaval Jashwantlal SOLANKI, Ahmedabad (IN); Sudhir Ganpatbhai DHANANI, Ahmedabad (IN)

(73) Assignee: CADILA HEALTHCARE LIMITED, Sarkhej-Gandhinagar Hwy (IN)

(21) Appl. No.: 17/147,928

(22) Filed: Jan. 13, 2021

(30) Foreign Application Priority Data
Jan. 13, 2020 (IN) 202021001502

(51) Int. Cl. C07D 213/72

(52) U.S. Cl. CPC C07D 213/72

(57) ABSTRACT

The present invention relates to a process for the preparation of fosnetupitant. The invention further relates to an amorphous fosnetupitant chloride hydrochloride and process for the preparation thereof.

### CROSS REFERENCE TO THE APPLICATION

This Application claims the benefit to India Patent Application No. 202021001502 filed Jan. 13, 2020, the content of which is incorporated herein by reference.

### FIELD OF THE INVENTION

The field of the invention relates to a process for the preparation of active pharmaceutical ingredient and solid forms thereof. In particular, the present invention relates to a process for the preparation of fosnetupitant. The invention further relates to an amorphous fosnetupitant chloride hydrochloride and the process for preparation thereof.

### BACKGROUND OF THE INVENTION

The following discussion of the prior art is intended to present the invention in an appropriate technical context and allow its significance to be properly appreciated. Unless clearly indicated to the contrary, reference to any prior art in this specification should be construed as such art is widely known or forms part of common general knowledge in the field.

Fosnetupitant, a prodrug of netupitant, is an NK-1 receptor antagonist useful in chemotherapy induced nausea and vomiting and can be represented as follows,

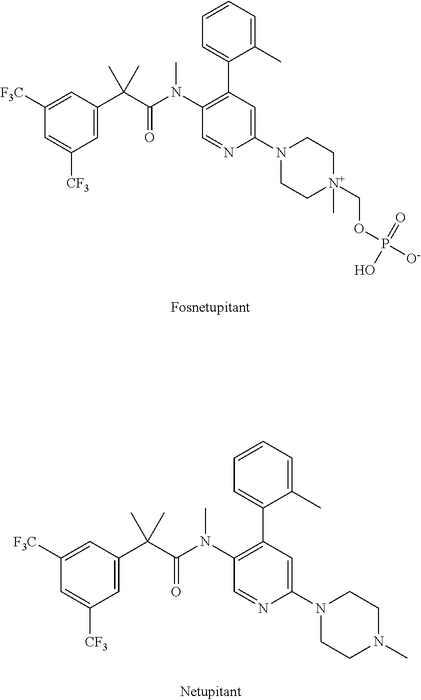 [SMILES: Cc1ccccc1-c1cc(N2CC[N+](C)(COP(=O)([O-])O)CC2)ncc1N(C)C(=O)C(C)(C)c1cc(C(F)(F)F)cc(C(F)(F)F)c1 ; Cc1ccccc1-c1cc(N2CCN(C)CC2)ncc1N(C)C(=O)C(C)(C)c1cc(C(F)(F)F)cc(C(F)(F)F)c1]

U.S. Pat. No. 9,403,772 B2 discloses the process for the preparation of Fosnetupitant, wherein netupitant and chloromethyl di-tert-butyl phosphate are reacted in a polar aprotic solvent.

International (PCT) Publication No. WO 2017/060338 discloses crystalline forms of Fosnetupitant chloride hydrochloride.

There exists a need to provide better alternative processes for preparation of Fosnetupitant and chloride hydrochloride salt thereof, which are easily scalable and eco-friendly.

### SUMMARY OF THE INVENTION

In one general aspect, there is provided a process for the preparation of Fosnetupitant, the process comprising:
(a) reacting netupitant and chloromethyl di-tert-butyl phosphate in one or more protic solvent; and
(b) obtaining Fosnetupitant.

In another general aspect, there is provided an amorphous Fosnetupitant chloride hydrochloride.

In another general aspect, there is provided a process for the preparation of amorphous Fosnetupitant chloride hydrochloride.

In another general aspect, there is provided a fosnetupitant chloride hydrochloride prepared by the process of present invention having purity of 98% w/w or more by percentage of HPLC.

### BRIEF DESCRIPTION OF THE DRAWINGS

FIG. 1: X-ray powder diffraction pattern of amorphous fosnetupitant chloride hydrochloride

### DETAILED DESCRIPTION OF THE INVENTION

The aforementioned general and further specific aspects of the invention are fulfilled by the description of the invention provided herein after.

Unless specifically described otherwise, the terms ‘reacting’, ‘treating’ and ‘condensing’ are generally interchangeable and are used in their ordinary meaning as they are used in the field of the invention, unless defined specifically otherwise.

Unless specifically described otherwise, the terms ‘isolating’, ‘obtaining’ and ‘purifying’ are generally interchangeable and include but not specifically limited to extraction, evaporation, crystallization, filtration, recrystallization, centrifugation, decantation or chromatographic operations.

In one general aspect, there is provided a process for the preparation of fosnetupitant, the process comprising:
(a) reacting netupitant and chloromethyl di-tert-butyl phosphate in one or more protic solvent; and
(b) obtaining Fosnetupitant.

In general, the protic solvent can be selected from a group comprising methanol, ethanol, propanol, isopropanol, n-butanol, t-butanol, pentanol, hexanol, heptanol, decanol, and dodecanol.

In general, Netupitant and chloromethyl di-tert-butyl phosphate can be reacted in the presence of a base in one or more protic solvents.

In general, the base comprises of an organic base or an inorganic base. The organic base can be selected from the group comprising ammonia, methylamine, ethylamine, t-butylamine, dimethylamine, diethylamine, diisopropylamine, trimethylamine, triethylamine, diisopropylmethylamine, diisopropylethylamine and tributylamine. The inorganic base can be selected from a group comprising sodium hydroxide, potassium hydroxide, lithium hydroxide, potassium carbonate, sodium carbonate, lithium carbonate, caesium carbonate, sodium bicarbonate, potassium bicarbonate, sodium methoxide, and potassium t-butoxide.

In general, Fosnetupitant can be prepared by reacting netupitant and chloromethyl di-tert-butyl phosphate in one or more protic solvents followed by treatment with an acid.

In general, Fosnetupitant can be prepared by reacting netupitant and chloromethyl di-tert-butyl phosphate in one or more protic solvents in the presence of a base, followed by treatment with an acid.

In general, the compound of Formula I can be isolated, if desired, after completion of the reaction between netupitant and chloromethyl di-tert-butyl phosphate, and before the treatment with an acid,

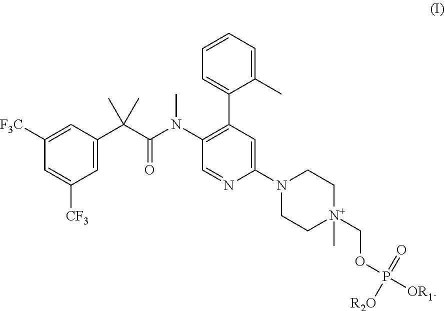

Wherein, R₁ and R₂ are independently negatively charged, H or t-Bu, provided at least one of the R₁ and R₂ is t-Bu.

In general, the compound of Formula I on treatment with an acid results in Fosnetupitant.

In general, the acid can be organic acid or inorganic acid. The inorganic acid can be selected from the group comprising hydrochloric acid, hydrobromic acid, hydroiodic acid, sulfuric acid, nitric acid, phosphoric acid, and boric acid. The organic acid can be selected from the group comprising acetic acid, formic acid, trifluoroacetic acid, citric acid, lactic acid, tartaric acid, benzoic acid, p-methylbenzoic acid, methanesulfonic acid, ethanesulfonic acid, benzenesulfonic acid, and p-toluenesulfonic acid.

In another general aspect, there is provided a process for the preparation of fosnetupitant, the process comprising:
(a) reacting netupitant and chloromethyl di-tert-butyl phosphate in one or more protic solvents to obtain a compound of Formula I; and
(b) obtaining fosnetupitant by the treatment of the compound of Formula I with an acid.

In another general aspect, there is provided a process for the preparation of fosnetupitant, the process comprising:
(a) reacting netupitant and chloromethyl di-tert-butyl phosphate in one or more protic solvents in the presence of a base to obtain a compound of Formula I; and
(b) obtaining fosnetupitant by the treatment of the compound of Formula I with an acid.

In general, the Fosnetupitant obtained can further be converted to its salt of Formula II,

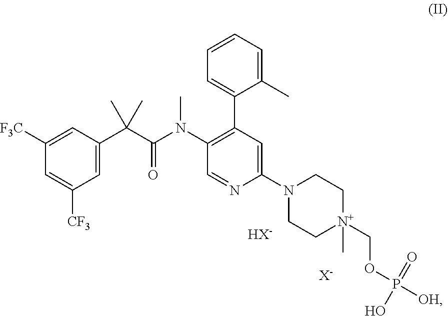

wherein, each X is independently an anionic counter ion especially to fosnetupitant chloride hydrochloride, wherein both the X are Cl in Formula II.

In another general aspect, there is provided a process for the preparation of a fosnetupitant salt of Formula II, the process comprising:
(a) reacting netupitant and chloromethyl di-tert-butyl phosphate in one or more protic solvents; and
(b) obtaining fosnetupitant salt of Formula II by the treatment with an acid.

In another general aspect, there is provided a process for the preparation of a fosnetupitant salt of Formula II, the process comprising:
(a) reacting netupitant and chloromethyl di-tert-butyl phosphate in a protic solvent to obtain a compound of Formula I; and
(b) obtaining fosnetupitant salt of Formula II by treatment of the compound of Formula I with an acid.

In another general aspect, there is provided a process for the preparation of fosnetupitant chloride hydrochloride, the process comprising:
(a) reacting netupitant and chloromethyl di-tert-butyl phosphate in a protic solvent; and
(b) obtaining fosnetupitant chloride hydrochloride by treatment with a hydrogen chloride source.

In another general aspect, there is provided a process for the preparation of fosnetupitant chloride hydrochloride, the process comprising:
(a) reacting netupitant and chloromethyl di-tert-butyl phosphate in a protic solvent to obtain a compound of Formula I; and
(b) obtaining fosnetupitant chloride hydrochloride by treatment of the compound of Formula I with a hydrogen chloride source.

In general, the source of hydrogen chloride can be selected from hydrogen chloride (HCl) gas, hydrochloric acid, aqueous hydrochloric acid and hydrogen chloride dissolved in a solvent such as methanol-HCl, ethanol-HCl, isopropanol-HCl, ethylacetate-HCl, isopropyl acetate-HCl, acetone-HCl, and ethylene dichloride-HCl. In general, any reagent or combination of reagents liberating HCl can also be provided as a source of hydrogen chloride.

In another general aspect, there is provided an amorphous fosnetupitant chloride hydrochloride.

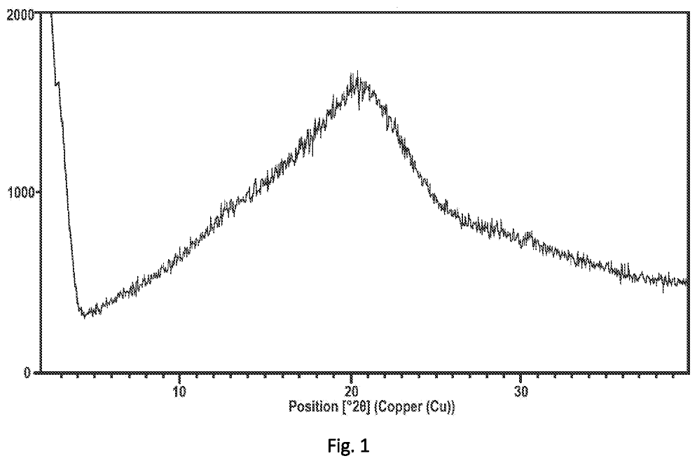

The amorphous fosnetupitant chloride hydrochloride can be characterised by an x-ray powder diffraction pattern substantially same as depicted in FIG. 1.

In another general aspect, there is provided a process for the preparation of amorphous fosnetupitant chloride hydrochloride.

The amorphous fosnetupitant chloride hydrochloride can be obtained by removal of solvent from a solution comprising fosnetupitant chloride hydrochloride either by spray drying, evaporation at reduced pressure or lyophilization. In particular, spray drying may be used.

The solution comprising fosnetupitant chloride hydrochloride can be obtained during the course of the reaction and/or process for the preparation of fosnetupitant chloride hydrochloride and/or purification of fosnetupitant chloride hydrochloride; or it can be prepared by dissolving fosnetupitant chloride hydrochloride in one or more solvents.

The amorphous fosnetupitant chloride hydrochloride can be obtained by solvent evaporation or evaporation at reduced pressure from a solution of fosnetupitant chloride hydrochloride in an alcoholic solvent or a ketonic solvent or a nitrile solvent, or mixtures thereof.

The amorphous fosnetupitant chloride hydrochloride can be obtained by spray drying of a solution of fosnetupitant chloride hydrochloride in an alcoholic solvent or a ketonic solvent or a nitrile solvent.

In general, the alcoholic solvent can be selected from one or more of methanol, ethanol, n-propanol, isopropanol, n-butanol, i-butanol, t-butanol, pentanol, hexanol, and heptanol. The ketonic solvent can be selected from one or more of acetone, methyl ethyl ketone, methyl isobutyl ketone, and diethyl ketone. The nitrile solvent can be selected from one or more of acetonitrile, benzonitrile, and butyronitrile.

In another general aspect, there is provided a fosnetupitant chloride hydrochloride prepared by the process of present invention having purity of 98% w/w or more by percentage of HPLC. In particular, the fosnetupitant chloride hydrochloride having a purity of 99% w/w, more particularly, a purity of 99.5% w/w or more by area percentage of HPLC.

The present invention is further illustrated by following reaction examples which are provided merely to be representative of the invention and do not limit the scope of it.

The product(s) obtained may further be converted to any other physical forms thereof which includes but not specifically limited to salt(s), solvate(s), hydrate(s), co-crystal(s) and solid dispersion(s) in either crystalline or amorphous forms.

The product(s) obtained may further be dried additionally to achieve desired level of moisture and/or residual solvents.

### EXAMPLES

### Example-1

### Preparation of Fosnetupitant Chloride Hydrochloride

In a 500 mL round bottom flask, 134.12 g Chloromethyl di-tert-butyl phosphate was added to a stirring mixture of 35 g trimethylamine and 100 g Netupitant in 650 mL t-butanol at 25-30° C. and stirred at 75-80° C. for 16-18 hours. The resultant mixture was cooled to 35-40° C. and 21 g of saturated solution of HCl in t-butanol was slowly added. The temperature was raised to 65-70° C. and 1000 mL ethyl acetate was added slowly and stirred for 10-20 min. The reaction mixture was cooled to 25-30° C., filtered, washed with ethyl acetate and dried to obtain fosnetupitant as its chloride hydrochloride salt.

### Example-2

### Preparation of Fosnetupitant Chloride Hydrochloride

A: In a 500 mL round bottom flask, 54 g Chloromethyl di-tert-butyl phosphate was added to a stirring mixture of 35 g trimethylamine and 100g Netupitant in 650 mL t-butanol at 25-30° C. and stirred at 75-80° C. for 16-18 h and the solvent was removed at reduced pressure. 500 mL acetonitrile was added to the residue, heated to 65-70° C., 300 mL diisopropylether was added and stirred for 10-20 min. The resultant mass was cooled to 25-30° C., filtered, washed with acetonitrile/diisopropylether mixture and dried to obtain the compound of Formula I (wherein one of R₁ and R₂ is negative charge and the other is t-Bu).

B: In a 250 mL round bottom flask, 1 g of HCl 4M in methanol was added slowly to the stirring mixture of the compound of Formula I in acetone (5 ml) at 25-30° C. The resultant solid was filtered, washed with Acetone (2 mL) and purified by additional slurry in acetone (10 mL) and filter and wash with acetone (2 mL) and dried to obtain fosnetupitant as its chloride hydrochloride salt.

### Example-3

### Preparation of Fosnetupitant Chloride Hydrochloride

A: In a round bottom flask, 134.12 g Chloromethyl di-tert-butyl phosphate was added to a stirring mixture of 41.80 g Potassium Carbonate and 100 g Netupitant in 500 mL t-butanol at 25-30° C. and stirred at 75-80° C. for 12 h and the solvent was removed at reduced pressure. 200 mL acetonitrile was added to the residue, heated to 50-60° C., and the solvent was removed at reduced pressure followed by further addition of 1000 mL acetonitrile. 1000 mL diisopropylether was added to reaction mixture and stirred for 30-40 min. The resultant mass was cooled to 25-30° C., filtered, washed with mixture of acetonitrile and diisopropylether, and dried the wet product under reduced pressure. Charged the dried material and methanol (400 ml) into round bottom flask stir for 50-70 min. and filtered the mass through filter pad followed by wash the pad with methanol (100 mL). Water (2500 mL) was added to the filtrate and the resultant solid was filtered, washed with water and dried to obtain Formula I.

B: In a round bottom flask, 49 g of a saturated solution of HCl in a 100 mL of acetone was added slowly to the stirring mixture of 100 g of compound of Formula I in 1400 mL acetone at 10-15° C. and stirred the reaction mixture to 25-35° C. for 48 hours. The resultant solid was filtered, washed with mixture of acetone followed by n-heptane and dried to obtain fosnetupitant as its chloride hydrochloride salt.

### Example-4

### Preparation of Amorphous Fosnetupitant Chloride Hydrochloride

A solution of 100 g fosnetupitant chloride hydrochloride in 1500 mL methanol was subjected to spray drying under following conditions to obtain amorphous fosnetupitant chloride hydrochloride.

| Parameter | Value Range | |
|---|---|---|
| Needle size | 0.5-0.7 | mm |
| Atomization pressure | 0.8 to 1.5 | Kg/cm² |
| Inlet temperature | 75-80° | C. |
| Outlet temperature | 70-75° | C. |
| Feed pump flow rate | 1-4 | ml/min |
| Aspirator flow rate | 50-100 | Nm³/hr. |
| Atomization pressure | 0.7-1.8 | Kg/cm² |
| Vacuum | (−45)-(+10) | mmwc |

### Example-5

### Preparation of Amorphous Fosnetupitant Chloride Hydrochloride

In a 500 mL round bottom flask, 100 g fosnetupitant chloride hydrochloride was stirred in mixture of 200 mL methanol and 100 mL dioxane at 40-45° C. to obtain a clear solution. The solvent was removed at reduced pressure to obtain amorphous fosnetupitant chloride hydrochloride.

While the present invention has been described in terms of a few specific aspects, modifications and equivalents thereof, in light of teaching and disclosure of the present invention, which are apparent of the skilled artisan, are to be construed as included within the scope of the invention.

We claim:

1. A process for the preparation of fosnetupitant, the process comprising:
(a) reacting netupitant with chloromethyl di-tert-butyl phosphate in one or more protic solvents; and
(b) obtaining fosnetupitant.

2. The process according to claim 1, wherein the fosnetupitant is converted to one or more salts of formula II, 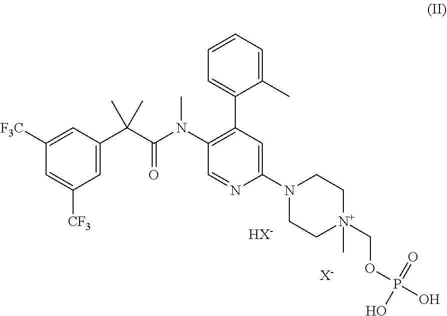
the process comprising treating fosnetupitant with an acid.

3. The process according to claim 2, wherein the fosnetupitant salt is fosnetupitant chloride hydrochloride, prepared by the process comprising:
(a) reacting netupitant and chloromethyl di-tert-butyl phosphate in one or more protic solvents; and
(b) obtaining fosnetupitant chloride hydrochloride by treatment with a hydrogen chloride source.

4. The process according to claim 2, further optionally comprising isolating compound of formula I, 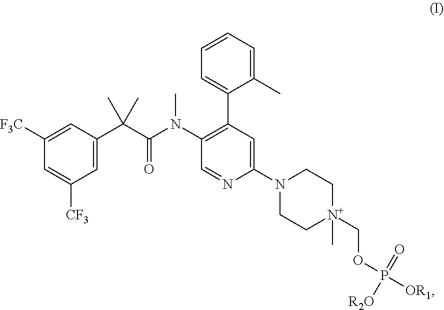
wherein, R₁ and R₂ are independently negatively charged, H or t-Bu, provided at least one of the R₁ and R₂ is t-Bu, before treatment with an acid.

5. The process according to claim 1, wherein the protic solvent comprises one or more of methanol, ethanol, propanol, isopropanol, n-butanol, t-butanol, pentanol, hexanol, heptanol, decanol, dodecanol, or mixtures thereof.

6. The process according to claim 1, wherein the reaction of netupitant with chloromethyl di-tert-butyl phosphate is performed in the presence of a base.

7. The process according to claim 1, wherein the method of obtaining fosnetupitant in step (b) comprises one or more of extraction, evaporation, crystallization, filtration, recrystallization, centrifugation, decantation, and chromatographic operations.

8. The process according to claim 6, wherein the base comprises one or more of ammonia, methylamine, ethylamine, t-butylamine, dimethylamine, diethylamine, diisopropylamine, trimethylamine, triethylamine, diisopropylmethylamine, diisopropylethylamine, tributylamine, sodium hydroxide, potassium hydroxide, lithium hydroxide, potassium carbonate, sodium carbonate, lithium carbonate, caesium carbonate, sodium bicarbonate, potassium bicarbonate, sodium methoxide, and potassium t-butoxide.

9. The process according to claim 2, wherein the acid comprises one or more of hydrochloric acid, hydrobromic acid, hydroiodic acid, sulfuric acid, nitric acid, phosphoric acid, boric acid, acetic acid, formic acid, trifluoroacetic acid, citric acid, lactic acid, tartaric acid, benzoic acid, p-methylbenzoic acid, methanesulfonic acid, ethanesulfonic acid, benzenesulfonic acid, and p-toluenesulfonic acid.

10. The process according to claim 3, wherein the source of hydrogen chloride in step (b) comprises one or more of hydrogen chloride (HCl) gas, hydrochloric acid, aqueous hydrochloric acid and hydrogen chloride dissolved in a solvent selected from methanol, ethanol, isopropanol, ethylacetate, isopropyl acetate, acetone, and ethylene dichloride.

11. An amorphous fosnetupitant chloride hydrochloride having a purity of 98% w/w or more by percentage of HPLC.

12. A process for the preparation of amorphous fosnetupitant chloride hydrochloride, the process comprising:
(a) providing a solution of fosnetupitant chloride hydrochloride in one or more of an alcoholic solvent, a ketonic solvent, a nitrile solvent, and mixtures thereof; and
(b) removing the solvent from the solution to obtain the amorphous fosnetupitant chloride hydrochloride.

13. The process according to claim 12, wherein the alcoholic solvent comprises one or more of methanol, ethanol, n-propanol, isopropanol, n-butanol, isobutanol, t-butanol, pentanol, hexanol, and heptanol, the ketonic solvent comprises one or more of acetone, methyl ethyl ketone, methyl isobutyl ketone, and diethyl ketone, and the nitrile solvent comprises one or more of acetonitrile, benzonitrile, and butyronitrile.

14. The process according to claim 12, wherein the removal of the solvent in step (b) comprises of spray drying, evaporation at reduced pressure or lyophilization.
